(19) United States
(12) Patent Application Publication
(10) Pub. No.: US 2025/0163378 A1
(43) Pub. Date: May 22, 2025

(54) CELL MICROPARTICLES CAPABLE OF TARGETING Folr2+ MACROPHAGES AND METHOD FOR PREPARING SAME

(71) Applicant: Tongji Hospital affiliated to Tongji Medical College of Huazhong University of Science & Technology, Wuhan (CN)

(72) Inventors: Rui Zeng, Wuhan (CN); Gang Xu, Wuhan (CN); Ying Yao, Wuhan (CN); Wenhui Liao, Wuhan (CN); Guoli Li, Wuhan (CN); Qian Yang, Wuhan (CN); Chang Xu, Wuhan (CN); Yanping Lin, Wuhan (CN)

(21) Appl. No.: 18/731,871

(22) Filed: Jun. 3, 2024

(30) Foreign Application Priority Data
Nov. 16, 2023 (CN) 202311531011.8

(51) Int. Cl. C12N 5/0786

(52) U.S. Cl. CPC C12N 5/0645

(57) ABSTRACT

The present disclosure relates to a method for preparing cell microparticles capable of targeting Folr2+ macrophages and the prepared cell microparticles, and the method includes the following steps: S1: culturing macrophages to obtain a macrophage culture; S2: extracting cell microparticles from the macrophage culture. By extracting cell microparticles from macrophages and coupling folic acid molecules on the surface thereof, the present disclosure allows the resulting cell microparticles to target Folr2 bound to the surface of macrophages and accurately deliver drugs such as cyclophosphamide, clodronate, doxorubicin, etc. to the site of kidney injury, thereby efficiently killing nephropathogenic Folr2+ macrophages and reducing systemic nonspecific distribution. The cell microparticles of the present disclosure have the advantages of homing targeting, ease of engineering, low cytotoxicity, and low immunogenicity.

### CROSS-REFERENCE TO RELATED APPLICATION

The present application claims priority from Chinese Patent Application No. 202311531011.8 filed on Nov. 16, 2023, the contents of which are incorporated herein by reference in their entirety.

### TECHNICAL FIELD

The present disclosure belongs to the field of renal injury treatment, and more particularly, to cell microparticles capable of targeting Folr2+ macrophages and a method for preparing the same.

### BACKGROUND ART

Macrophages play an important role in a variety of inflammatory and fibrotic diseases, affecting the development and outcome of a variety of chronic diseases. Studies have shown that there are pathogenic macrophage subsets of disease. Among them, macrophages expressing Folic acid receptor 2 (Folr2) as activated macrophages are closely related to the progression of various chronic diseases such as anti-GBM nephritis, rheumatoid arthritis, systemic lupus erythematosus, hepatocellular carcinoma, skin fibrosis, and pulmonary fibrosis. We speculate that Folr2 may be a target to break through the treatment of many chronic diseases.

However, there is no safe and effective treatment strategy for Folr2+ macrophages. Traditional treatment strategies for macrophages, such as chlorophosphate liposome clearance of macrophages, which clears all kinds of macrophages, lacks specificity, cannot achieve the effect of accurate treatment, and has obvious side effects. One study showed that cytotoxic drugs targeting Folr2+ macrophages could effectively inhibit rat anti-GBM nephritis, unfortunately, this drug is limited in molecular characteristics and safety and is not suitable for clinical application.

Cell microparticles, also known as exosomes, are nanovesicles secreted by natural cells, small lipid bilayer particles carrying nucleic acids, proteins, and lipids, have good biocompatibility and cell absorption capacity, have high stability in body fluids, and can evade the recognition of the immune system. Macrophage-derived microparticle vesicles can be loaded with diagnostic molecules or chemotherapeutic drugs and delivered to macrophages in vitro and in vivo for diagnostic or therapeutic purposes.

Thus, cell microparticles can be used to prepare biopharmaceuticals that target Folr2+ macrophages.

In the present disclosure, the macrophage-derived microparticulate vesicles are selected to take advantage of their natural affinity for macrophages to increase their specificity by anchoring the relevant to target homing to the macrophage and achieving the effect of intervention on specific macrophages.

Cell microparticles, also known as exosomes, are nanovesicles secreted by most natural cells, small lipid bilayer particles carrying nucleic acids, proteins, and lipids, have good biocompatibility and cell absorption capacity, have high stability in body fluids, and can evade the recognition of the immune system. Macrophage-derived microparticle vesicles can be loaded with diagnostic molecules or chemotherapeutic drugs and delivered to macrophages in vitro and in vivo for diagnostic or therapeutic purposes. In the present disclosure, the macrophage-derived microparticle vesicles are selected to take advantage of their natural affinity for macrophages to increase their specificity by anchoring the relevant to target homing to the macrophage and achieving the effect of intervention on specific macrophages.

Folr2 protein is localized to the cell membrane surface. Folr2 binds to folic acid and reduced folic acid derivatives and mediates the delivery of 5-methyltetrahydrofolic acid and folic acid analogs into the cell interior. It has a high affinity for folic acid and folic acid analogs at neutral pH.

### SUMMARY OF THE INVENTION

To solve the above problems, the present disclosure provides a method for preparing cell microparticles capable of targeting pathogenic Folr2+ macrophages, including the step of extracting the microparticles from the macrophages.

In a specific embodiment, the method includes the following steps:
S1: culturing macrophages to obtain a macrophage culture;
S2: extracting cell microparticles from the macrophage culture.

In a specific embodiment, in S1, the macrophages are cultured using RPMI1640 medium supplemented with DSPE-PEG-FA and the culture time is not shorter than 3 days.

In a specific embodiment, the macrophage is Raw264.7.

In a specific embodiment, S2 includes the following steps:
S21: subjecting the macrophage culture to ultraviolet irradiation;
S22: extracting the cell microparticles from the macrophage culture after the ultraviolet irradiation.

In a specific embodiment, S21 includes the following steps:
S211: resuspending the macrophage culture with serum-free medium to obtain a cell suspension;
S212: subjecting the cell suspension to ultraviolet irradiation for 1 h; and S213: allowing the cell suspension to stand at 37° C. for over 10 h.

In a specific embodiment, S22 includes the following steps:
S221: performing low-speed centrifugation on the cell suspension to obtain a supernatant containing cell microparticles;
S222: subjecting the supernatant containing cell microparticles to high-speed centrifugation to obtain a cell microparticle precipitate precipitate; and
S223: resuspending the cell microparticle precipitate to obtain the cell microparticle.

In a specific embodiment, the step of co-incubating the cell microparticles with a drug is further included.

In a specific embodiment, the drug is a chlorophosphate.

The present disclosure also provides cell microparticles capable of targeting Folr2+ macrophages prepared by the methods described above.

By extracting cell microparticles from macrophages and coupling folic acid molecules on the surface thereof, the present disclosure allows the resulting cell microparticles to target Folr2 bound to the surface of macrophages and accurately deliver drugs such as cyclophosphamide, clodronate, doxorubicin, etc. to the site of injury, thereby efficiently killing nephropathogenic Folr2+ macrophages and reducing systemic nonspecific distribution. The cell microparticles of the present disclosure have the following advantages: 1) homing targeting: Macrophage-derived microparticles can home to natural macrophages and to accurately recognize and efficiently target specific macrophages while reducing uptake by other non-targeted cells. 2) ease of engineering: Through click chemistry, bioorthogonal, and other means, cell microparticle surface is easy to modify a variety of targeting ligands and functional molecules to further improve their targeting and functionality. 3) Low cytotoxicity and low immunogenicity.

### BRIEF DESCRIPTION OF THE DRAWINGS

FIG. 1 shows animal intervention experiments with drug-free cell microparticles. Wherein, A is the intervention experimental scheme diagram of a mouse model with early renal injury; B is in vitro organ fluorescence images of mice with early renal injury; C is a statistical graph of the uptake of fluorochrome-labeled IR780 cell microparticles by Folr2+ macrophages in various organs of mice with early injury; D is a statistical graph of the uptake of cell microparticle by Folr2+ macrophages in the left injured kidney and right healthy kidney of mice with early injury; E is the intervention experimental scheme diagram of a mouse model with advanced renal injury; F is in vitro organ fluorescence images of mice with advanced renal injury; G is a statistical graph of the uptake of microparticles by Folr2+ macrophages in various organs of mice with advanced renal injury; H is a statistical graph of the uptake of microparticles by Folr2+ macrophages in organs per body weight of mice with advanced renal injury

FIG. 2 shows animal intervention experiments with drug-loaded cell microparticles. Wherein, A is the intervention experimental scheme diagram of a mouse model with renal injury; B is a statistical graph of kidney weight and body weight of mice in each group; C is the statistical graph of body weight of mice in each group; D is the scheme diagram of flow cytometry and the statistical graph of the total amount of immune cells, macrophages, Folr2-negative and positive macrophages in the kidney of mice in each group; E is the PAS staining map of renal tissue and the statistical graph of the number of injured renal tubules in each group; F is the Masson staining map of kidney tissue and the statistical graph of Masson positive area in each group of mice; G is the immunofluorescence staining map of kidney tissue and the statistical graph of fibronectin positive area in each group of mice.

### DETAILED DESCRIPTION OF THE INVENTION

In order that the objects and advantages of the present disclosure become more apparent, a more particular description of the invention will be rendered by reference to the embodiments thereof which are illustrated in the appended drawings. It should be understood that the particular embodiments described herein are illustrative only and are not limiting. Furthermore, the technical features involved in the various embodiments of the present disclosure described below can be combined with each other as long as they do not conflict with each other.

### 1. Preparation of Macrophage Microparticles

Macrophages were cultured in vitro and Raw264.7 was selected. Macrophages were cultured routinely in RPMI1640 medium supplemented with DSPE-PEG-FA (where FA is a folic acid molecule and DSPE-PEG is a membrane-associated molecular complex) to a concentration of 25 ug/ml for more than 3 consecutive days. After culturing to a certain amount, macrophage microparticles are extracted.

The specific procedures for extracting macrophage microparticles are as follows: the culture dish supernatant was discarded, the bottom of culture dish was carefully scraped with cell scraper, 2 ml serum-free culture medium was added to clean the culture dish, after cleaning, the cells of three to five culture dishes were combined into one culture dish, the cover of culture dish was open, to irradiate under the ultraviolet light for 1 h in sterile operation platform, and then it was placed into 37° C. incubator overnight.

The next day, all cell suspensions were collected and centrifuged in a horizontal centrifuge at 1000 g for 10 min. The supernatant was then placed in a 1.5 ml EP tube, and centrifuged at 14,000 rpm for 30 min. Macrophage microparticles were then resuspended in the appropriate amount of PBS solution and stored at −80° C. for later use.

Microparticle concentration was quantified by measuring microparticle vesicle protein content by lysing the microparticle vesicles. Macrophage microparticles were then co-incubated with different concentrations of drug solutions, after overnight in the refrigerator at 4° C., it was washed with PBS solution, centrifuged at 14,000 rpm for 30 min, washed 3 times repeatedly, and resuspended to obtain drug-loaded macrophage microparticles.

2. Animal Intervention Experiments with Drug-Free Macrophage Microparticles

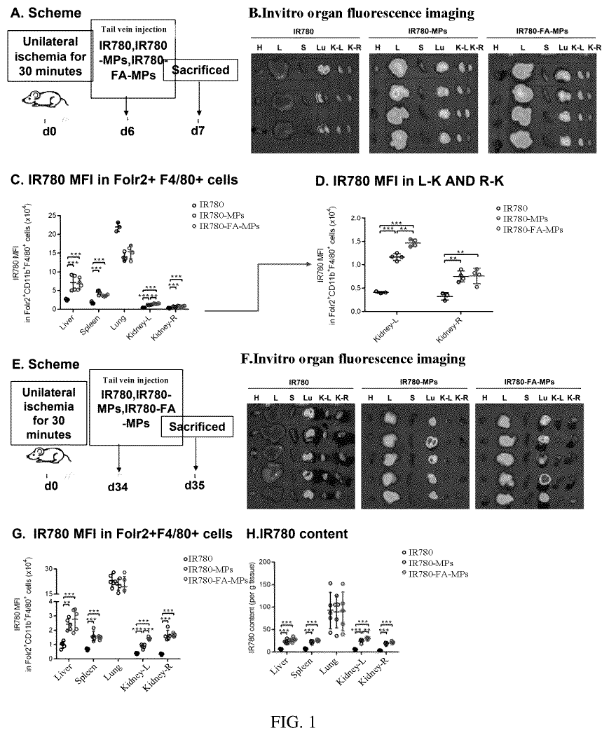

As shown in FIG. 1A, the left kidney of the mouse was subjected to unilateral ischemia-reperfusion for 30 min to obtain a mouse model with left kidney injury.

On Day 6, free dye IR780, IR780-labeled blank vesicle microparticles (IR780-MPs), and IR780-labeled vesicle microparticles with folic acid molecules (IR780-FA-MPs) were injected into the tail vein. After 24 h, the mice were sacrificed and the heart, liver, spleen, lung, and left and right kidneys were removed.

The results of in vitro fluorescence imaging of various organs are shown in FIG. 1B, and there is no significant difference in the uptake of the material in the heart, liver, spleen, and lung; However, it was found that the injured kidney (K-L) ingested more vesicle microparticles than the unaffected kidney (K-R).

Further, we performed a flow cytometric analysis of individual organs to examine the uptake of vesicle microparticles by Folr2+ macrophages. Results as shown in FIG. 1C, the uptake of both IR780-MPs and IR780-FA-MPs was higher in each organ than in the free dye group, indicating that the microparticles improve drug delivery efficiency. Furthermore, there was no significant difference in uptake between IR780-MPs and IR780-FA-MPs groups in the heart, liver, spleen, and lung. However, the injured side kidneys had more uptake of IR780-FA-MPs 7 days after kidney injury (FIG. 1D).

Then we did the same verification on the late stage of renal injury (FIGS. 1E-G) and reached the same conclusion, that is, FA-MPs have a certain targeting to the injured kidney.

3. Animal Intervention Experiments with Drug-Loaded Macrophage Microparticles

Chlorophosphate was selected as the loading drug. Referring to the clearance of macrophages by chlorophosphate liposomes in previous studies, we set low concentration group of 100 μg (BIR+CI@FA-MPs-low) and high concentration group of 200 μg (BIR+Cl@FA-MPs-high), and control group (BIR+PBS).

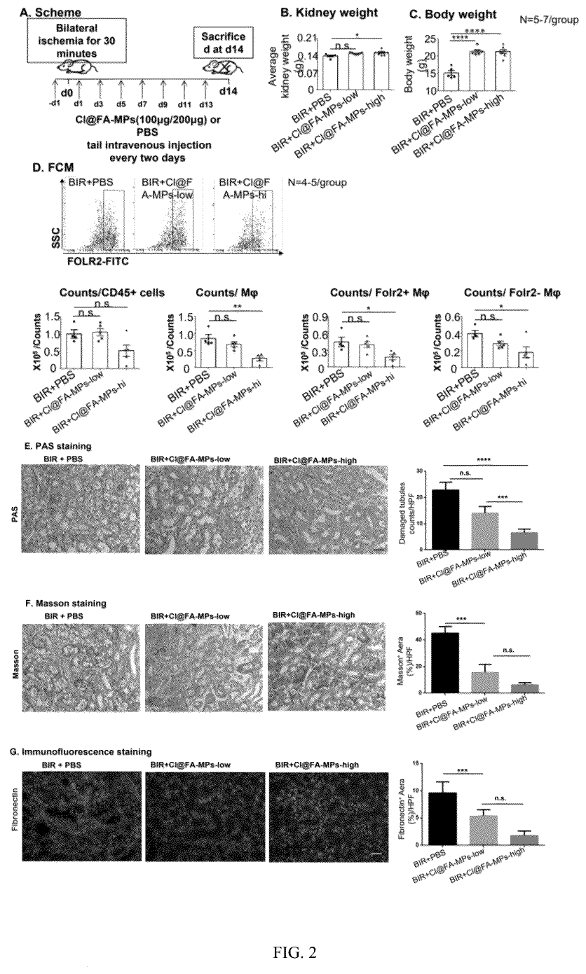

Mice with bilateral renal ischemia-reperfusion received tail vein injection (FIG. 2A) one day prior to modeling and then once every other day. Mice were sacrificed on day 14. The weight and kidney weight of mice were weighed (FIGS. 2B-C). At the same time, the kidneys of mice were detected by flow cytometry (FIG. 2D) to detect the changes of Folr2+ macrophages. The results showed that compared with the control group, Folr2+ macrophages in the kidneys of mice in the high-dose group were significantly reduced. Pathological staining of the kidneys (Passion, Masson, Immunofluorescence) showed (FIG. 2E) that the renal injury in the two groups loaded with cell microparticles was significantly lower than that in the control group, and the renal injury in the high dose group was significantly lower than that in the high concentration group. The drug loading of vesicle microparticles was shown to be effective on a specific subset of macrophages in the kidney.

It will be appreciated by persons skilled in the art that the above description is of preferred embodiments of the present disclosure and is not intended to limit the present disclosure. Thus, it is intended that the present disclosure covers the modifications, equivalents, and alternatives falling within the spirit and scope of the present disclosure.

1. A method for preparing cell microparticles capable of targeting Folr2+ macrophages, comprising the step of extracting the microparticles from the macrophages.

2. The method according to claim 1, comprising the following steps:
S1: culturing macrophages to obtain a macrophage culture;
S2: extracting cell microparticles from the macrophage culture.

3. The method according to claim 2, wherein in S1, the macrophages are cultured using RPMI1640 medium supplemented with DSPE-PEG-FA and the culture time is not shorter than 3 days.

4. The method according to claim 2, wherein the macrophage cell is Raw264.7.

5. The method according to claim 2, wherein S2 comprises the following steps:
S21: subjecting the macrophage culture to ultraviolet irradiation;
S22: extracting the cell microparticles from the macrophage culture after ultraviolet irradiation.

6. The method according to claim 5, wherein S21 comprises the following steps:
S211: resuspending the macrophage culture with serum-free medium to obtain a cell suspension;
S212: subjecting the cell suspension to ultraviolet irradiation for 1 h; and
S213: allowing the cell suspension to stand at 37° C. for over 10 h.

7. The method according to claim 6, wherein S22 comprises the following steps:
S221: performing low-speed centrifugation on the cell suspension to obtain a supernatant containing cell microparticles;
S222: subjecting the supernatant containing cell microparticles to high-speed centrifugation to obtain a cell microparticle precipitate; and
S223: resuspending the cell microparticle precipitate to obtain the cell microparticle.

8. The method according to claim 1, further comprising the step of co-incubating the cell microparticles with a drug.

9. The method according to claim 8, wherein the drug is a chlorophosphate.

10. A cell microparticle capable of targeting nephropathogenic Folr2+ macrophages, prepared by the method according to claim 1.

11. A cell microparticle capable of targeting nephropathogenic Folr2+ macrophages, prepared by the method according to claim 2.

12. A cell microparticle capable of targeting nephropathogenic Folr2+ macrophages, prepared by the method according to claim 3.

13. A cell microparticle capable of targeting nephropathogenic Folr2+ macrophages, prepared by the method according to claim 4.

14. A cell microparticle capable of targeting nephropathogenic Folr2+ macrophages, prepared by the method according to claim 5.

15. A cell microparticle capable of targeting nephropathogenic Folr2+ macrophages, prepared by the method according to claim 6.

16. A cell microparticle capable of targeting nephropathogenic Folr2+ macrophages, prepared by the method according to claim 7.

17. A cell microparticle capable of targeting nephropathogenic Folr2+ macrophages, prepared by the method according to claim 8.

18. A cell microparticle capable of targeting nephropathogenic Folr2+ macrophages, prepared by the method according to claim 9.
